(19) United States
(12) Patent Application Publication
(10) Pub. No.: US 2026/0335638 A1
(43) Pub. Date: Oct. 29, 2026

(54) DEVICE FOR TEMPERATURE MEASUREMENT IN ELECTRODE ARRAY

(71) Applicant: FIELDCURE CO., LTD., Seoul (KR)

(72) Inventors: Jin Young HONG, Seoul (KR); Dong Jun LEE, Seoul (KR); Woo Jung KANG, Yonginsi (KR); Jong Hyun KIM, Seoul (KR)

(73) Assignee: FIELDCURE CO., LTD., Seoul (KR)

(21) Appl. No.: 19/117,655

(22) PCT Filed: Sep. 13, 2023
(86) PCT No.: PCT/KR2023/013743

§ 371 Date: Apr. 2, 2025

(30) Foreign Application Priority Data
Oct. 7, 2022 (KR) 10-2022-0128261
Aug. 31, 2023 (KR) 10-2023-0115095

(51) Int. Cl. A61N 1/36; A61N 1/02; A61N 1/04

(52) U.S. Cl. CPC A61N 1/36002; A61N 1/025; A61N 1/0476; A61N 1/0492; A61N 1/36034

(57) ABSTRACT

A device for temperature measurement in an electrode array according to one embodiment of the present invention is attached to the surface of an object to transmit an electric field to the object. The device may comprise: a plurality of electrodes (1100) which are mounted to the electrode array to transmit the electric field to the object; a plurality of temperature measurement modules (1200) for measuring the temperature between the plurality of electrodes (1100) and the surface of the object; and a digital control unit (1300) which is connected to the plurality of temperature measurement modules (1200), transmits digital signals for controlling each of the temperature measurement modules (1200), and receives response signals from each of the temperature measurement modules (1200). According to one embodiment of the present invention, the device has the effect of reducing the complexity of a circuit and interface cables for measuring the temperature of each of electrodes in an electric field therapy electrode array.

### CROSS-REFERENCE TO RELATED APPLICATION

This application is an International Application No. PCT/KR2023/013743 filed on Sep. 13, 2023, which claims priority from Korean Patent Application No. 10-2023-0115095 filed on Aug. 31, 2023 and Korean Provisional Patent Application No. 10-2022-0128261 filed on Oct. 7, 2022, the entire contents of which are incorporated herein by reference.

### TECHNICAL FIELD

The present disclosure relates to an apparatus for measuring temperature in an electrode array, and more particularly, to an apparatus with a minimized interface cable to the electrode array for temperature measurement.

### BACKGROUND ART

The tumor treating fields (TTFields), which induce cell death of tumors by delivering alternating electric fields to tumors, is a proven cancer treatment that has been approved by the FDA for relapsed glioblastoma and newly diagnosed glioblastoma.

The tumor treating fields system consists of a generator (alternating current signal generator), a divider, and a plurality of electrode arrays to deliver a therapeutic electric field to a target area of the subject.

According to U.S. Pat. Nos. 8,715,203 and 8,764,675, each electrode array consists of a plurality of electrode elements that are attached to the surface of the subject. A typical electrode array for glioblastoma consists of a 3×3 matrix structure of capacitive coupled nine individual electrodes, each electrode has a temperature sensor to measure the temperature between the electrodes and the skin. By the divider, the generator is connected with two pairs of electrode arrays which cover two directions orthogonal to each other. The divider connects the first pair of electrode arrays for a certain period of time and conducts current, and then selects a pair of second electrode arrays for a certain period of time. The current generated by the generator is connected to a pair of selected electrode arrays by the divider, and flows along a current path formed by the electrodes of the first electrode array, the skin of the subject to which the electrodes are attached, and the body of the subject, the skin of the subject to which the electrodes of the second electrode array are attached and the second electrode array. At this time, a therapeutic electric field of more than a certain intensity is delivered to the target area of the cancer cell, and the therapeutic electric field interferes with or delays the division of the dividing cancer cell to destroy the cancer cells.

At this time, the electric current flowing between the electrode and the skin generates heat, so it is necessary to measure the temperature by an additional temperature sensor to prevent skin burns caused by this heat. Optimization methods have been disclosed to control the intensity of the current by pausing the system or modifying the current waveform to avoid the risk of the subject being burned when the measurement value of each temperature sensor exceeds a certain temperature (e.g., 41 degrees).

The interface cable between the electrode array and the divider is very complex, consisting of a conductor for the current path, an analog signal line for outputting the temperature measurement value to the generator, and additional lines for the temperature measurement circuit. Alternating currents of hundreds of kHz over 1 A can cause changes in the magnetic field around the conductor for the current path, and these changes can cause unnecessary interference with the temperature analog output line, thus causing a problem with accurate temperature measurement.

In order to solve this problem, U.S. Registered U.S. Pat. No. 11,097,101 and Korea Laid-open Patent No. 10-2020-0008542 use analog multiplexes, constant current sources, analog-to-digital converters (ADCs), and controllers to sequentially measure temperature within an electrode array and digitally output the measured temperature values. U.S. Laid-open Patent No. 2022-0193404 extends this method to use two analog multiplexes to select a temperature sensor by matrix switching method, and to control the current waveform of the generator based on the measured temperature value. U.S. Laid-open Patent No. 2022-0257927 discloses a method for switching each electrode array based on the temperature of four or more electrode arrays.

However, despite these different approaches, there is still a need for additional temperature measurement circuits and communication lines to control them. Therefore, there is a need for a method that can sequentially measure the temperature of each electrode without additional circuit elements.

### DISCLOSURE OF INVENTION

### Technical Problem

In order to solve the problem, the present disclosure provides an apparatus for measuring temperature in an electrode array in which the interface cable with the electrode array is minimized.

### Solution to Problem

In order to accomplish the aforementioned object, the present disclosure provides an apparatus for measuring temperature in an electrode array attached to the surface of a subject to deliver an electric field. The apparatus may include a plurality of electrodes 1100 mounted in the electrode array to deliver an electric field to the subject; a plurality of temperature measurement modules 1200 for measuring the temperature between the plurality of electrodes 1100 and the surface of the subject; and a digital control unit 1300 connected to the plurality of temperature measurement modules 1200, transmitting a digital signal to control the temperature measurement modules 1200, and receiving a response signal from each temperature measurement module 1200.

Here, the plurality of temperature measurement modules may be connected to the digital control unit in cascade, and each temperature measurement module may include a temperature sensor 1210 for measuring temperature; a transducer 1220 for converting the measured temperature value into digital signals; a receiver 1230 for receiving digital signals; a transmitter 1240 for transmitting digital signals; and a temperature measurement module control unit 1250 for controlling the temperature sensor 1210, the transducer 1220, the receiver 1230 and the transmitter 1240.

The temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230.

The temperature measurement module control unit 1250 may combine the received measurement signal and the temperature value signal converted from the measured temperature value to form a single data sequence, and provide the single data sequence outside the temperature measurement module 1200.

Here, the received measurement signal in the temperature measurement module 1200 may include converted temperature value signals from at least one adjacent temperature measurement module in addition to a temperature measurement command.

The digital control unit 1300 and the plurality of temperature measurement modules 1200 may be connected in cascade to analyze the response signal received by the digital control unit 1300 thereby identifying position information and the temperature value of each temperature measurement module 1200.

Here, the plurality of temperature measurement modules may be connected in parallel to the digital control unit 1300, and each temperature measurement module may include a temperature sensor 1210 for measuring temperature; a transducer 1220 for converting the measured temperature value into digital signals; a receiver 1230 for receiving the digital signals; a transmitter 1240 for transmitting the digital signals; and a temperature measurement module control unit 1250 for controlling the temperature sensor 1210, the transducer 1220, the receiver 1230 and the transmitter 1240.

The temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230.

The temperature measurement module control unit 1250 may combine the received measurement signal and the temperature value signal converted from the measured temperature value to form a single data sequence, and provide the single data sequence outside the temperature measurement module 1200.

Here, the digital control unit 1300 may have a plurality of communication ports, each communication port may represent the position information of one temperature measurement module 1200, and the temperature value of each position may be identified by analyzing the response signal received by each communication port.

Here, the digital control unit 1300 may set a temperature measurement cycle, monitor the temperature measured by the temperature measurement module in real time according to the temperature measurement cycle, and provide position information and the monitored temperature measurement value of the temperature measurement module to the outside if the monitored temperature measurement exceeds a predetermined temperature range

Here, the digital control unit 1300 may set a temperature measurement cycle, monitor the temperature measured by the temperature measurement module in real time according to the temperature measurement cycle, and provide a rate of change of the monitored temperature measurement value, the monitored temperature measurement value and position information of the temperature measurement module to the outside if the rate of change exceeds a predetermined rate of change range

Here, the electrode array may be formed of a flexible substrate, and attached to the subject flexibly along a curved surface of the subject.

Here, the inventive concept may include a power line communication unit (not shown in drawings) by which the digital control unit 1300 transmits digital data received from the temperature measurement module 1200 using power line communication.

Here, the inventive concept may include a high-frequency alternating current signal generator (not shown in drawings) generating an alternating current voltage of 50~500 kHz in order to deliver an electric field to the subject by the plurality of electrodes 1100.

In order to accomplish the aforementioned object, the present disclosure provides an apparatus for measuring temperature in an electrode array according to another embodiment of the inventive concept, which is attached to the surface of a subject to deliver an electric field. The apparatus may include a plurality of electrodes 1100 mounted in the electrode array to deliver an electric field to the subject; a plurality of temperature measurement modules 1200 for measuring the temperature between the plurality of electrodes and the surface of the subject; and a digital control unit 1300 connected to the plurality of temperature measurement modules 1200, transmitting a digital signal to control the temperature measurement modules 1200, and receiving a response signal from each temperature measurement module 1200.

Each temperature measurement module 1200 may include a temperature sensor 1210 for measuring temperature; a transducer 1220 for converting the measured temperature value into digital signals; a receiver 1230 for receiving the digital signals; a transmitter 1240 for transmitting the digital signals; and a temperature measurement module control unit 1250 for controlling the temperature sensor 1210, the transducer 1220, the receiver 1230 and the transmitter 1240,

The temperature sensor 1210 is assigned a unique identifier, and the temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230.

The temperature measurement module control unit 1250 may combine the unique identifier, the received measurement signal and the temperature value signal converted from the measured temperature value to form a single data sequence, and provide the single data sequence outside the temperature measurement module 1200.

### Advantageous Effects of Invention

The interface cable between the electrode array and the divider for conventional tumor treating fields requires numerous lines, including a power line for a current path to flow current by each individual electrode and for the temperature measurement circuit to measure the temperature between the electrode and the skin, and an analog signal line to send the temperature measurement value.

In addition, due to the influence of the magnetic field caused by the current path, errors in temperature measurements and unnecessary noise are bound to be included.

In order to solve these problems, the inventive concept discloses a method and an apparatus consisting of a daisy-chain of temperature sensors equipped in an electrode array, sequentially digitally measuring and outputting these measurement values.

According to an apparatus for measuring temperature in an electrode array according to an embodiment of the inventive concept, the interface cable between the electrode array and the divider may consist of only a conductor for the current path and two conductors for the power supply of the temperature sensor by outputting the measured temperature value using a power line communication protocol. This has the advantage of reducing the complexity of circuits and interface cables for measuring the temperature of each electrode in an array of electric field therapy electrodes.

### BRIEF DESCRIPTION OF DRAWINGS

FIG. 1 is a schematic view illustrating an apparatus for measuring temperature in an electrode array according to an embodiment of the inventive concept.

FIG. 2 is a schematic view illustrating an apparatus for measuring temperature in an electrode array according to another embodiment of the inventive concept.

FIG. 3 is a schematic view illustrating a temperature measurement module of an apparatus for measuring temperature in an electrode array according to an embodiment of the inventive concept.

FIG. 4 is a block diagram of a temperature sensor equipped with a serial interface and supporting a daisy-chain structure in an apparatus for measuring temperature in an electrode array according to an embodiment of the inventive concept.

FIG. 5 is a schematic view of a host controller for transmitting commands to a plurality of temperature sensors configured in a daisy-chain and receiving temperature measurement data in an apparatus for measuring temperature in an electrode array according to an embodiment of the inventive concept.

FIG. 6 is a schematic view illustrating a host controller for serial communication with a generator or a divider in an apparatus for measuring temperature in an electrode array according to an embodiment of the inventive concept.

FIG. 7 is a schematic view illustrating a host controller for power line communication with a generator or a divider in an apparatus for measuring temperature in an electrode array according to an embodiment of the inventive concept.

FIG. 8 is a schematic view illustrating a temperature sensor, a communication controller, a cable and a plurality of electrodes composing typical 3×3 electrode pads in an apparatus for measuring temperature in an electrode array according to an embodiment of the inventive concept.

### MODE FOR THE INVENTION

Embodiments of the inventive concept will be described more fully hereinafter with reference to the accompanying drawings and let those skilled in the art implement easily. The inventive concept may be implemented in various forms and not limited to the following some embodiments.

In denoting reference numerals to elements of each drawing, the same reference numerals denote the same elements though the elements are shown in different drawings. In describing embodiments of the inventive concepts, descriptions for universally known elements or functions may be left out when the descriptions make obscure essential points of the inventive concepts.

If there is referred that a part is connected to another part, it includes a case of ‘connecting directly’ with each other by interposing another device as well as a case of ‘connecting electrically’ with each other. In the addition, if there is referred that any part ‘include(s)’ a component, it means that other components are not excluded but further includes another component unless special opposite statement is described.

If there is referred that a part ‘on’ another part, it is on the part or another part may be interposed therebetween. If there is referred that a part ‘directly on’ another part, another part should not be interposed therebetween.

Terms of a first, a second, a third will be used to describe various parts, elements, regions, layers and/or sections, but not limited to these. These terms may be used only to distinguish a part, component, area, layer, or section from another, component, area, layer, or section. Accordingly, the first part, component, area, layer, or section described hereinafter may be referred to as a second part, component, area, layer, or section to the extent that it is not beyond the scope of the inventive concept.

The professional terms will be used hereinafter in order to mention only specific embodiment, but it does not intend to limit the inventive concept. The singular forms used here include plural forms, unless the phrases clearly indicate the opposite meaning. The meaning of “including” in the specification specifies a particular characteristic, domain, integer, step, action, element, and/or component, and does not exclude the presence or addition of other properties, areas, integers, phases, motions, elements, and/or components.

Terms such as “under” and “on” that refer to relative space may be used to more easily describe the relationship of one part to another part shown in the drawing. These terms may be intended to include any other meaning or behavior of the device being used, along with the intended meaning in the drawing. For example, if you flip the device in the drawing, some parts described as being “under” the other parts are described as being “on” the other parts. Thus, the illustrative term “under” encompasses both of under and on. The device may be rotated 90 degrees or at different angles, and the terms used to describe relative space are interpreted accordingly.

Although not otherwise defined, all terms including technical and scientific terminology used herein may have the same meanings as generally understood by those skilled in the art. Commonly used dictionary-defined terms are further construed to have meanings consistent with the relevant technical literature and current disclosure, and are not interpreted in an ideal or highly formal sense unless they are defined.

Embodiments of the inventive concept will be described more fully hereinafter with reference to the accompanying drawings and let those skilled in the art implement easily. The inventive concept may be implemented in various forms and not limited to the following some embodiments.

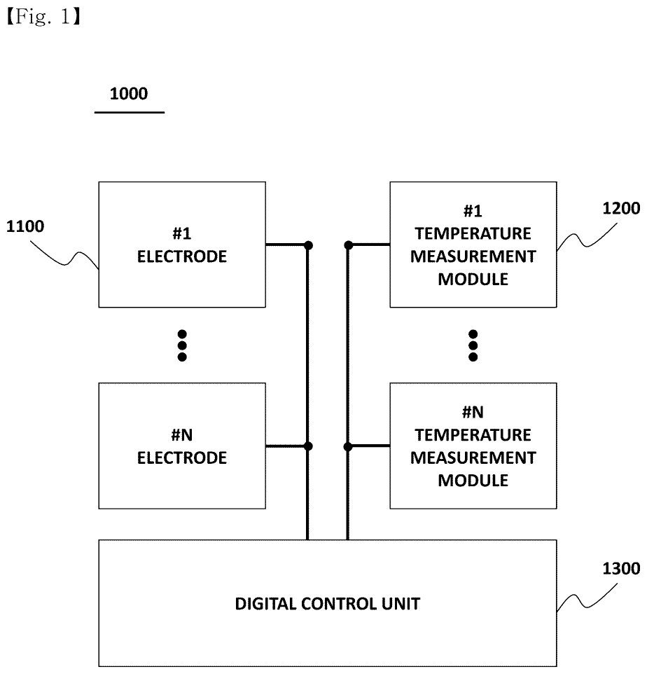

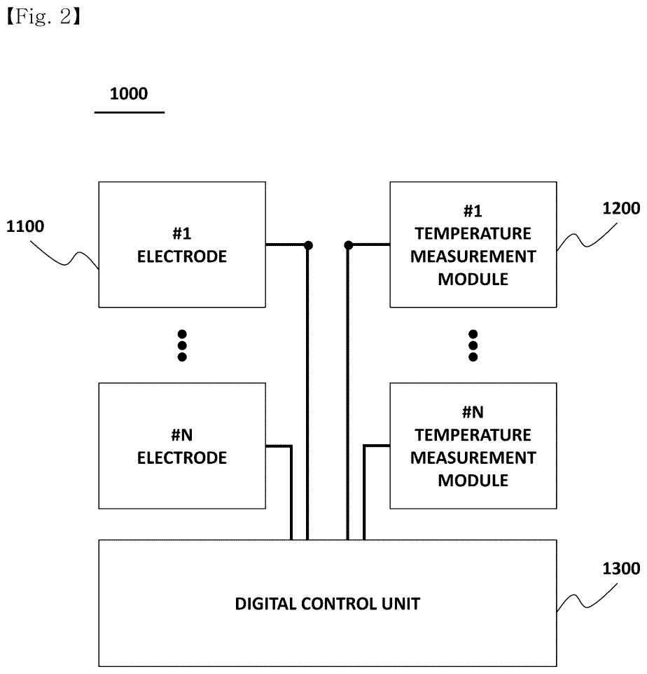

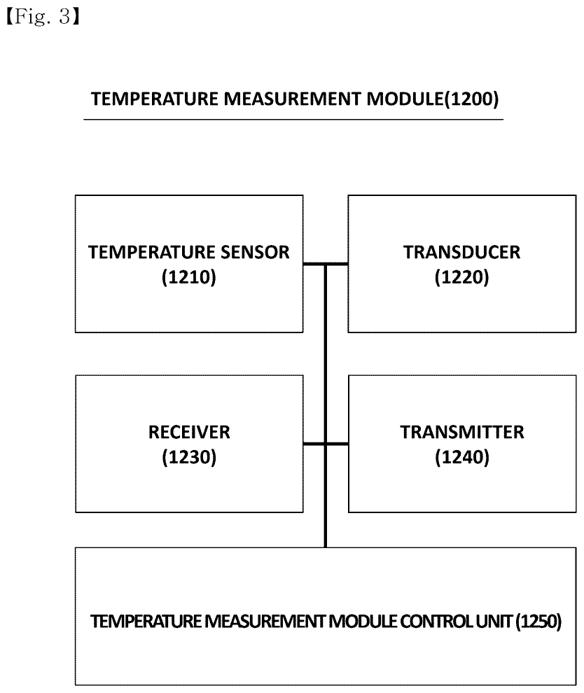

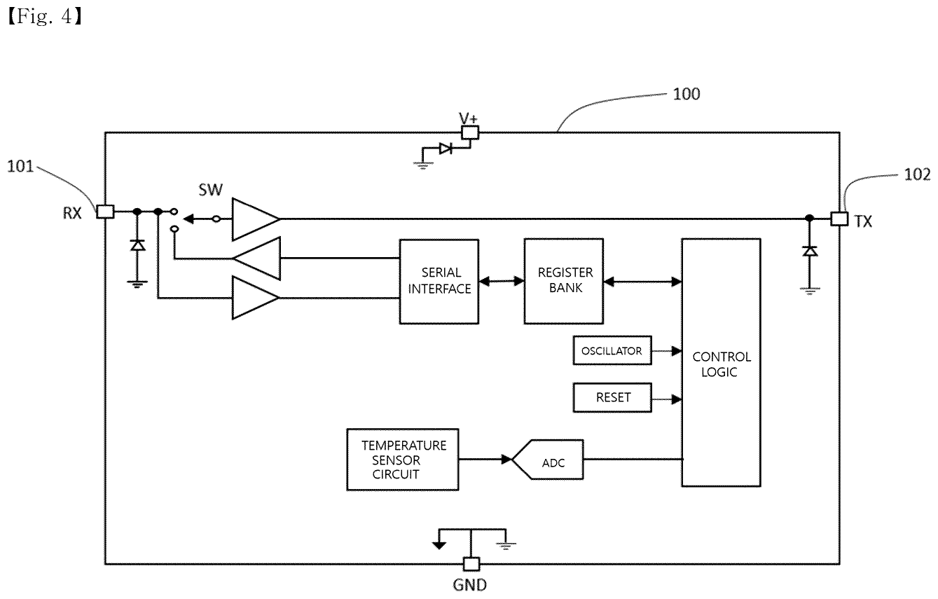

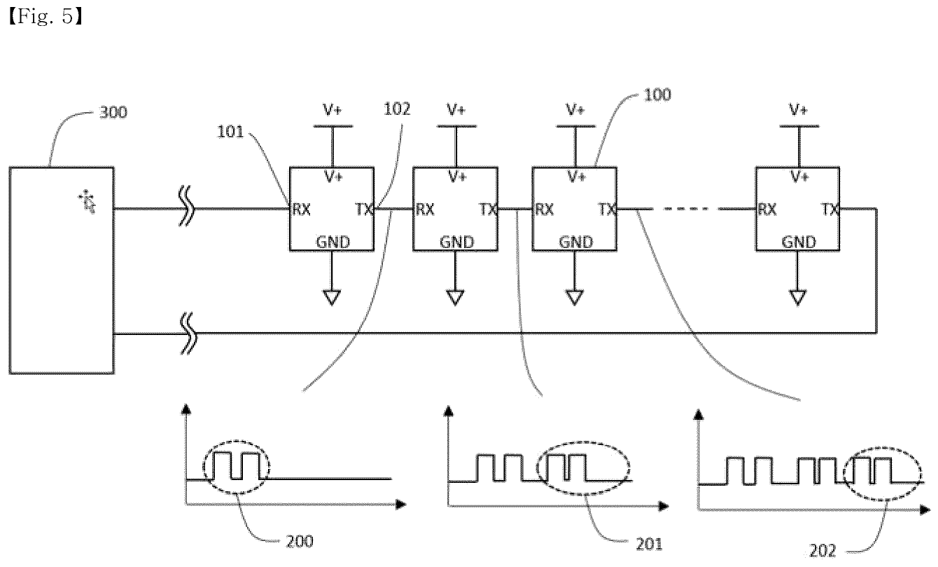

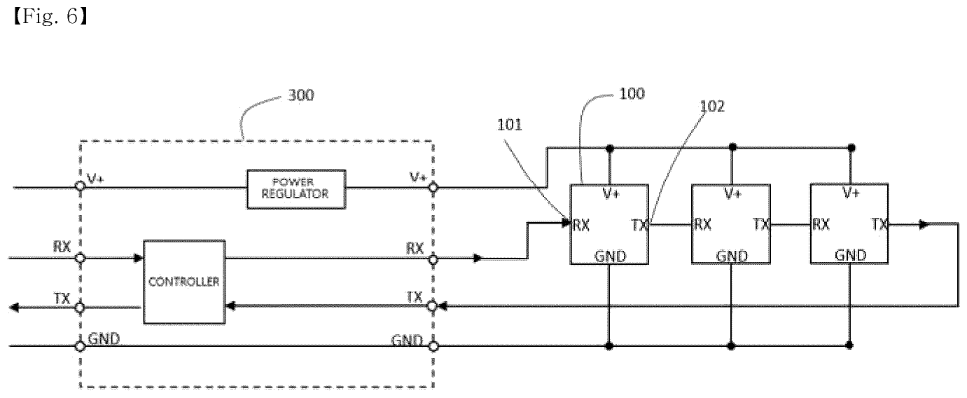

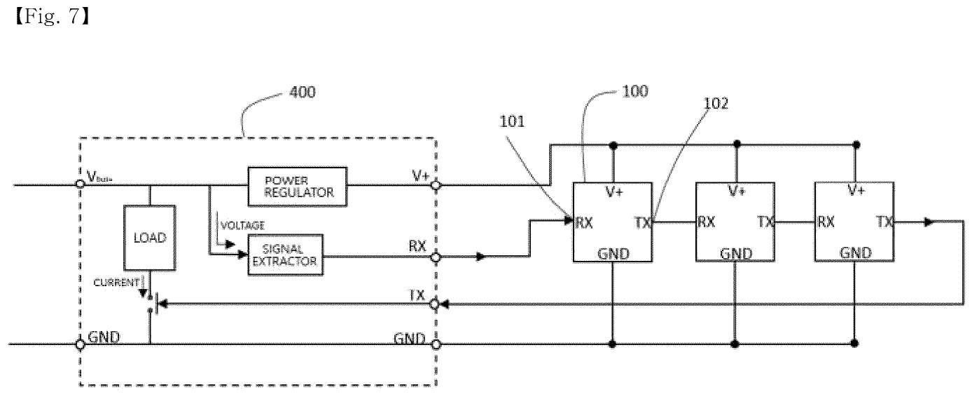

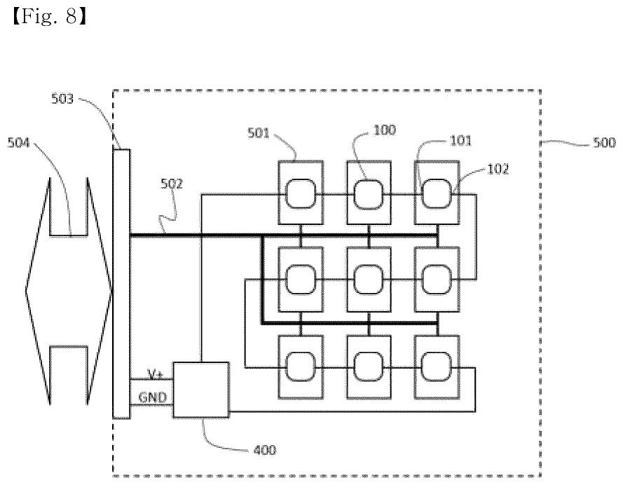

FIG. 1 is a schematic view illustrating an apparatus for measuring temperature in an electrode array according to an embodiment of the inventive concept. FIG. 2 is a schematic view illustrating an apparatus for measuring temperature in an electrode array according to another embodiment of the inventive concept. FIG. 3 is a schematic view illustrating a temperature measurement module of an apparatus for measuring temperature in an electrode array according to an embodiment of the inventive concept. FIG. 4 is a block diagram of a temperature sensor equipped with a serial interface and supporting a daisy-chain structure in an apparatus for measuring temperature in an electrode array according to an embodiment of the inventive concept. FIG. 5 is a schematic view of a host controller for transmitting commands to a plurality of temperature sensors configured in a daisy-chain and receiving temperature measurement data in an apparatus for measuring temperature in an electrode array according to an embodiment of the inventive concept. FIG. 6 is a schematic view illustrating a host controller for serial communication with a generator or a divider in an apparatus for measuring temperature in an electrode array according to an embodiment of the inventive concept. FIG. 7 is a schematic view illustrating a host controller for power line communication with a generator or a divider in an apparatus for measuring temperature in an electrode array according to an embodiment of the inventive concept. FIG. 8 is a schematic view illustrating a temperature sensor, a communication controller, a cable and a plurality of electrodes composing typical 3×3 electrode pads in an apparatus for measuring temperature in an electrode array according to an embodiment of the inventive concept.

Referring to FIGS. 1 to 8 together, an apparatus for measuring temperature in an electrode array attached to the surface of a subject to deliver an electric field may include a plurality of electrodes 1100 mounted in the electrode array to deliver an electric field to the subject; a plurality of temperature measurement modules 1200 for measuring the temperature between the plurality of electrodes 1100 and the surface of the subject; and a digital control unit 1300 connected to the plurality of temperature measurement modules 1200, transmitting a digital control signal to the temperature measurement modules 1200, and receiving a response signal from each temperature measurement module 1200.

The electrode array may be used to deliver an electric field to the subject, attached to the surface of the subject, and equipped with a plurality of electrodes 1100.

The plurality of electrodes 1100 may be mounted on the electrode array to deliver an electric field to the subject. The plurality of electrodes 1100 may be mounted in various arrangement forms to the electrode array in consideration of the efficiency of electric field transmission.

The plurality of temperature measurement modules 1200 may measure the temperature between the plurality of electrodes 1100 and the surface of the subject.

The digital control unit 1300 may be connected to the plurality of temperature measurement modules 1200 to transmit a digital signal for controlling the temperature measurement modules 1200, and receive response signals of each temperature measurement module 1200.

In the apparatus for measuring temperature in the electrode array 1000 according to an embodiment of the inventive concept, the plurality of temperature measurement modules 1200 may be connected in parallel to the digital control unit 1300, and each temperature measurement module 1200 may include a temperature sensor 1210 for measuring temperature; a transducer 1220 for converting the measured temperature value into digital signals; a receiver 1230 for receiving the digital signals; a transmitter 1240 for transmitting the digital signals; and a temperature measurement module control unit 1250 for controlling the temperature sensor 1210, the transducer 1220, the receiver 1230 and the transmitter 1240

In addition, the temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230.

The temperature measurement module control unit 1250 may combine the received measurement signal and the temperature value signal converted from the measured temperature value to form a single data sequence, and provide the single data sequence outside the temperature measurement module 1200.

Referring to FIGS. 1 and 3 together, the plurality of the temperature measurement modules 1200 may be connected with the digital control unit 1300 in cascade, and the temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230. The temperature measurement module control unit 1250 may combine the received measurement signal and the temperature value signal converted from the measured temperature value to form a single data sequence, and provide the single data sequence outside the temperature measurement module 1200.

Eventually, the digital control unit 1300 may receive the one data sequence, and it will be possible to grasp the measured temperature for each temperature measurement module 1200 among the plurality of temperature measurement modules 1200 by analyzing the one data sequence, and it will also be possible to identify which temperature measurement module 1200 measures the temperature.

The measurement signal received in the temperature measurement module 1200 may include converted temperature value signals from at least one adjacent temperature measurement module in addition to a temperature measurement command, and the digital control unit 1300 and the plurality of temperature measurement modules 1200 may be connected in cascade to analyze the response signal received by the digital control unit 1300 thereby identifying position information and the temperature value of each temperature measurement module 1200

In the apparatus for measuring temperature in the electrode array 1000 according to an embodiment of the inventive concept, the plurality of temperature measurement modules 1200 may be connected in parallel to the digital control unit 1300, and each temperature measurement module 1200 may include a temperature sensor 1210 for measuring temperature; a transducer 1220 for converting the measured temperature value into digital signals; a receiver 1230 for receiving the digital signals; a transmitter 1240 for transmitting the digital signals; and a temperature measurement module control unit 1250 for controlling the temperature sensor 1210, the transducer 1220, the receiver 1230 and the transmitter 1240.

The temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230.

The temperature measurement module control unit 1250 may combine the received measurement signal and the temperature value signal converted from the measured temperature value to form a single data sequence, and provide the single data sequence outside the temperature measurement module 1200.

In addition, the digital control unit 1300 may have a plurality of communication ports, and each communication port may represent the position information of one temperature measurement module 1200, and analyze the response signal received by each communication port to identify the temperature value of each position.

Referring to FIGS. 2 and 3 together, the plurality of the temperature measurement modules 1200 may be connected in parallel to the digital control unit 1300, and the temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230. The temperature measurement module control unit 1250 may combine the received measurement signal and the temperature value signal converted from the measured temperature value to form a single data sequence, and provide the single data sequence outside the temperature measurement module 1200.

In particular, the digital control unit 1300 may have a plurality of communication ports, and each communication port may represent the position information of one temperature measurement module 1200, and the temperature value of each position may be identified by analyzing the response signal received by each communication port.

Consequently, the digital control unit 1300 may easily determine which temperature measurement module 1200 corresponds to each communication port and the temperature value measured by each communication port when analyzing the received response signals through the plurality of communication ports connected in parallel to the digital control unit 1300.

On the other hand, the digital control unit 1300 may set a temperature measurement cycle, monitor the temperature measured by the temperature measurement module in real time according to the temperature measurement cycle, and provide position information and the monitored temperature measurement value of the temperature measurement module to the outside if the monitored temperature measurement exceeds a predetermined temperature range

In addition, the digital control unit 1300 may set a temperature measurement cycle, monitor the temperature measured by the temperature measurement module in real time according to the temperature measurement cycle, and provide a rate of change of the monitored temperature measurement value, the monitored temperature measurement value and position information of the temperature measurement module to the outside if the rate of change exceeds a predetermined rate of change range

Consequently, the digital control unit 1300 may set a temperature measurement cycle, monitor the temperature measured by the temperature measurement module in real time according to the temperature measurement cycle, i) provide the position information and the monitored temperature measurement value of the temperature measurement module to the outside if the monitored temperature measurement exceeds a predetermined temperature range, and ii) provide a rate of change of the monitored temperature measurement value, the monitored temperature measurement value and the position information of the temperature measurement module to the outside if the rate of change exceeds a predetermined rate of change range.

The electrode array of the apparatus for measuring temperature in the electrode array according to an embodiment of the inventive concept may be formed of a flexible substrate, and attached to the subject flexibly along a curved surface of the subject. That is, since the subject may not form a flat surface, the electrode array may be formed of a flexible substrate so that it can be adapted to the surface shape of the subject.

The apparatus for measuring temperature in the electrode array according to an embodiment of the inventive concept may include a power line communication unit (not shown in drawings) by which the digital control unit 1300 transmits digital data received from the temperature measurement module 1200 using power line communication.

The apparatus for measuring temperature in the electrode array according to an embodiment of the inventive concept may connect a generator (not shown in drawings) generating an alternating current voltage of 50~500 kHz in order to deliver an electric field to the subject by the plurality of electrodes 1100. Therefore, the apparatus for measuring temperature in the electrode array which is suggested in the inventive concept may be used universally for treatment methods other than the tumor treating fields in which the subject skin and the electrode array are in electrical contact to maintain the skin temperature within a certain temperature.

An apparatus for measuring temperature in an electrode array 1000 according to an embodiment of the inventive concept, which is attached to the surface of a subject to deliver an electric field, may include a plurality of electrodes 1100 mounted in the electrode array to deliver an electric field to the subject; a plurality of temperature measurement modules 1200 for measuring the temperature between the plurality of electrodes and the surface of the subject; and a digital control unit 1300 connected to the plurality of temperature measurement modules 1200, transmitting a digital control signal to the temperature measurement modules 1200, and receiving a response signal from each temperature measurement module 1200.

Each temperature measurement module may include a temperature sensor 1210 for measuring temperature; a transducer 1220 for converting the measured temperature value into digital signals; a receiver 1230 for receiving the digital signals; a transmitter 1240 for transmitting the digital signals; and a temperature measurement module control unit 1250 for controlling the temperature sensor 1210, the transducer 1220, the receiver 1230 and the transmitter 1240,

The temperature sensor 1210 is assigned a unique identifier, and the temperature measurement module control unit 1250 may measure the temperature by the temperature sensor 1210, convert the measured temperature value into a temperature value signal by the transducer 1220, and provide the converted temperature value signal by the transmitter 1240, in accordance with the measurement signal received by the receiver 1230.

The temperature measurement module control unit 1250 may combine the unique identifier, the received measurement signal and the temperature value signal converted from the measured temperature value to form a single data sequence, and provide the single data sequence outside the temperature measurement module 1200.

Consequently, each temperature sensor 1210 may be assigned a unique identifier, and the temperature measurement module control unit 1250 may combine the unique identifier, the received measurement signal and the temperature value signal to which the measured temperature value is converted to form a single data sequence, and then the one data sequence may be provided outside the temperature measurement module 1200, so that it can be determined utilizing the unique identifier which temperature measurement module measures the temperature value if analyzing the received data sequence.

On the other hand, from another point of view, the inventive concept relates to an electrode array for use with a generator (alternating current signal generator) that delivers a therapeutic electric field to the target area of the subject. The electrode array contains a plurality of electrode elements configured to be arranged on the subject. Each of the electrode elements is configured to attach to the subject's skin. A plurality of temperature sensors detects the temperature of the plurality of electrodes, and the electrode array includes a temperature sensor.

FIG. 4 is a block diagram of a temperature sensor that supports serial communication for measuring the temperature of each electrode in an electric field therapy electrode array. A temperature sensor 100 may have a built-in temperature sensor inside. The built-in temperature sensor may change the voltage with the temperature and the voltage may be converted into a digital value by an analog-to-digital converter. According to the commands received by the control logic, register bank, and serial interface, the received commands and data may be output by TX as they are, or the measured temperature value may be added to the received data and output by TX.

An example of a component suitable for this purpose is Texas Instrument's TMP144. The component is programmed to a unique interface address based on the position of the temperature sensor 100 after the initialization sequence and may respond to its own address individually. In addition, temperature may be measured sequentially in response to general commands that allow the user to read or write to all devices on the bus, without having to send individual addresses and commands to each device. Up to 16 temperature sensors can be daisy-chained.

FIG. 5 shows an example of transmitting and receiving temperature information by configuring a plurality of temperature sensors 100 in a daisy-chain by the host controller 300. When the host sends a command, the first temperature sensor 100 may add its temperature measurement value 200 to the data part and send it by the TX 102 port, and the second temperature sensor may receive the command and data received from RX and add its temperature measurement value 201 to the data part. In this daisy-chaining method, the temperature sensor may add its temperature measurement value to the received data and transmit by TX. The data transmitted from the last temperature sensor may be received by the host controller to complete the entire cycle.

FIG. 6 shows the structure of the host controller 300 for controlling a temperature sensor and sending measured temperature data to a generator or divider as an embodiment. The host controller may send the received temperature measurement value to the generator or divider by UART.

FIG. 7 shows the structure of the host controller 400 for controlling a temperature sensor and sending measured temperature data to a generator or divider as an embodiment. Here, the host controller may be structured for power line communication. Temperature measurement value obtained sequentially by a plurality of temperature sensors 100 configured in a daisy-chain may be converted into current by a load in the host controller and placed on a power line and transmit temperature information to a generator or divider.

FIG. 8 shows the structure of an electrode array 500 having a typical 3×3 electrode structure, and illustrates an embodiment in which a host controller 400 connected in cascade with nine temperature sensors 100 for measuring temperature is placed in the electrode array. The electrode array comprises a plurality of electrode 501 elements configured to be positioned to the subject. Each electrode attached to the subject's skin may be connected to conductor 502 which is an electric current path, and deliver a therapeutic electric field to the subject. The host controller 400 may communicate a temperature measurement value acquired by serial communication with a plurality of temperature sensors 100 that measure the temperature between the underside of each electrode and the skin with the divider or generator by connector 504 and connection line 503 that are connected to the generator or distributor.

The host controller of FIG. 8 may be configured in an electrode array or arranged on the divider side. Based on the temperature values of each electrode measured by a plurality of the temperature sensors consisting of daisy chains, the intensity of the electric current applied for electric field therapy can be adjusted.

Although the embodiment of the inventive concept has been described with reference to accompanying diagram, those skilled in the art will be understood that various other modifications of the disclosed embodiments will become apparent without modification of technical spirit or essential feature of the inventive concept. For example, those skilled in the art may change the material, size, etc. of each component according to the field of application, or combine or replace the embodiments disclosed and perform them in a form that is not clearly disclosed in the embodiment of the inventive concept, but this is also not beyond the scope of the inventive concept. Therefore, the embodiments described above are exemplary and should not be understood as being limited in all respects, and it should be said that these variations embodiments are included in the technical ideas described in claims of the inventive concept.
1000: Apparatus for measuring temperature in electrode array
1100: Electrodes #1 to #N
1200: Temperature measurement module
1210: T
1220: Transducer
1230: Receiver
1240: Transmitter
1250: Temperature measurement module control unit
1300: Digital control unit

1. An apparatus for measuring temperature in an electrode array attached to the surface of a subject for delivering an electric field to the subject comprising:
a plurality of electrodes arranged in the electrode array for delivering the electric field to the subject;
a plurality of temperature measurement modules for measuring the temperature between the electrodes and the surface of the subject; and
a digital control unit connected to the plurality of temperature measurement modules, transmitting a digital signal to control each temperature measurement module, and receiving a response signal from each temperature measurement module.

2. The apparatus for measuring temperature in an electrode array of claim 1, wherein the plurality of temperature measurement modules is connected to the digital control unit in cascade,
wherein each temperature measurement module comprises:
a temperature sensor for measuring temperature;
a transducer for converting the measured temperature value into digital signals;
a receiver for receiving digital signals;
a transmitter for transmitting digital signals; and
a temperature measurement module control unit for controlling the temperature sensor, the transducer, the receiver and the transmitter,
wherein the temperature measurement module control unit measures the temperature by the temperature sensor, converts the measured temperature value into a temperature value signal by the transducer, and provides the converted temperature value signal by the transmitter, in accordance with the measurement signal received by the receiver, and
wherein the temperature measurement module control unit combines the received measurement signal and the temperature value signal converted from the measured temperature value to form a single data sequence, and provides the single data sequence outside the temperature measurement module.

3. The apparatus for measuring temperature in an electrode array of claim 2, wherein the measurement signal received in the temperature measurement module comprises converted temperature value signals from at least one adjacent temperature measurement module in addition to a temperature measurement command, and
wherein the digital control unit and the plurality of temperature measurement modules are connected in cascade to analyze the response signal received by the digital control unit thereby identifying position information and the temperature value of each temperature measurement module.

4. The apparatus for measuring temperature in an electrode array of claim 1, wherein the plurality of temperature measurement modules are connected in parallel to the digital control unit,
wherein each of the temperature measurement modules comprises:
a temperature sensor for measuring temperature;
a transducer for converting the measured temperature value into digital signals
a receiver for receiving digital signals;
a transmitter for transmitting digital signals; and
a temperature measurement module control unit for controlling the temperature sensor the transducer, the receiver and the transmitter,
wherein the temperature measurement module control unit measures the temperature by the temperature sensor, converts the measured temperature value into a temperature value signal by the transducer, and provides the converted temperature value signal by the transmitter in accordance with the measurement signal received by the receiver, and
wherein the temperature measurement module control unit combines the received measurement signal and the temperature value signal converted from the measured temperature value to form a single data sequence, and provides the single data sequence outside the temperature measurement module.

5. The apparatus for measuring temperature in an electrode array of claim 4, wherein the digital control unit has a plurality of communication ports, and each of the communication ports represents position information of one temperature measurement module, and the temperature value of each position is identified by analyzing the response signal received by each of the communication ports.

6. The apparatus for measuring temperature in an electrode array of claim 3, wherein the digital control unit sets a temperature measurement cycle,
monitors the temperature measured by the temperature measurement module in real time according to the temperature measurement cycle, and
provides the position information and the monitored temperature measurement value of the temperature measurement module to the outside if the monitored temperature measurement exceeds a predetermined temperature range.

7. The apparatus for measuring temperature in an electrode array of claim 3, wherein the digital control unit sets a temperature measurement cycle,
monitors the temperature measured by the temperature measurement module in real time according to the temperature measurement cycle, and
provides a rate of change of the monitored temperature measurement value, the monitored temperature measurement value and the position information of the temperature measurement module to the outside if the rate of change of the temperature measurement value exceeds a predetermined rate of change range.

8. The apparatus for measuring temperature in an electrode array of claim 1, wherein the electrode array is formed of a flexible substrate, and attached to the subject flexibly along a curved surface of the subject.

9. The apparatus for measuring temperature in an electrode array of claim 1, further comprising:
a power line communication unit by which the digital control unit transmits digital data received from the temperature measurement module using power line communication.

10. The apparatus for measuring temperature in an electrode array of claim 1, further comprising:
a high-frequency alternating current signal generator generating an alternating current voltage of 50~500 kHz in order to deliver an electric field to the subject by the plurality of electrodes.

11. An apparatus for measuring temperature in an electrode array attached to the surface of a subject to deliver an electric field comprising:
a plurality of electrodes mounted in an electrode array to deliver an electric field to the subject;
a plurality of temperature measurement modules for measuring the temperature between the plurality of electrodes and the surface of the subject; and
a digital control unit connected to the plurality of temperature measurement modules, transmitting a digital signal to each temperature measurement module, and receiving a response signal from each temperature measurement module.
wherein each temperature measurement module comprises:
a temperature sensor for measuring the temperature;
a transducer for converting the measured temperature value into digital signals
a receiver for receiving digital signals;
a transmitter for transmitting digital signals; and
a temperature measurement module control unit for controlling the temperature sensor, the transducer, the receiver and the transmitter,
wherein the temperature sensor is assigned a unique identifier,
wherein the temperature measurement module control unit measures the temperature by the temperature sensor, converts the measured temperature value into a temperature value signal by the transducer, and provides the converted temperature value signal by the transmitter in response to the measurement signal received by the receiver, and
wherein the temperature measurement module control unit combines the unique identifier, the received measurement signal, and the temperature value signal converted from the measured temperature value to form a single data sequence, and provides the single data sequence outside the temperature measurement module.

12. The apparatus for measuring temperature in an electrode array of claim 5, wherein the digital control unit sets a temperature measurement cycle,
monitors the temperature measured by the temperature measurement module in real time according to the temperature measurement cycle, and
provides the position information and the monitored temperature measurement value of the temperature measurement module to the outside if the monitored temperature measurement exceeds a predetermined temperature range.

13. The apparatus for measuring temperature in an electrode array of claim 5, wherein the digital control unit sets a temperature measurement cycle,
monitors the temperature measured by the temperature measurement module in real time according to the temperature measurement cycle, and
provides a rate of change of the monitored temperature measurement value, the monitored temperature measurement value and the position information of the temperature measurement module to the outside if the rate of change of the temperature measurement value exceeds a predetermined rate of change range.
